(19) United States
(12) Patent Application Publication
(10) Pub. No.: US 2023/0076477 A1
(43) Pub. Date: Mar. 9, 2023

(54) IMAGING DEVICE AND IMAGING METHOD

(71) Applicant: SHIMADZU CORPORATION, KYOTO-SHI (JP)

(72) Inventor: Kazuyuki MATSUDA, KYOTO-SHI (JP)

(73) Assignee: SHIMADZU CORPORATION, KYOTO-SHI (JP)

(21) Appl. No.: 17/467,261

(22) Filed: Sep. 5, 2021

(51) Int. Cl. A61B 5/026; G06T 7/00; A61B 5/00

(52) U.S. Cl. CPC A61B 5/0261; G06T 7/0012; A61B 5/0071; A61B 5/742; G06T 2207/10064; G06T 2207/30104

(57) ABSTRACT

An imaging device and a method of imaging accurately decides a cause when pixel values of each pixel in a predetermined region of a fluorescence image varies with time. The imaging device, has an excitation light source, an imaging element that acquires a fluorescence image, an image storing element that stores the fluorescence image with time, a pixel value measurement element an average value calculation element and a first curve generation element that generates showing a time-course change of the average pixel value of the entire fluorescence image, a second curve generation element that generates showing a time-course change of the average pixel value of the region of interest.

### CROSS REFERENCE TO RELATED APPLICATIONS

This application relates to, but does not claim priority from, JP 2019-038527 filed Mar. 4, 2019 and published on Sep. 10, 2020, the entire contents of which are incorporated herein by reference.

### FIGURE SELECTED FOR PUBLICATION

FIG. 8.

### BACKGROUND OF THE INVENTION

### Field of the Invention

The present invention relates to an imaging device and a method of imaging.

### Description of the Related Art

A method called near-infrared fluorescence imaging is applied to an angiography in a surgery. According to the near-infrared fluorescence imaging, indocyanine green (ICG), which is a fluorochrome, is administered into an affected area. And when a near-infrared light having wavelength approximately 600 to 850 nm is irradiated as an excitation light to such an indocyanine green, indocyanine green emits a near-infrared fluorescence having approximately wavelength 750 to 900 nm. Such a fluorescence is imaged by an image pickup element capable of detecting the near-infrared light, and then the image thereof is displayed on a display such as a liquid crystal display panel and so forth. When such near-infrared fluorescence imaging is applied, such as blood vessels and lymphatic vessels existing approximately 20 mm below the body surface can be observed.

For example, the patent Document 1 discloses a method of acquiring a graph showing a time-course change of the fluorescence intensity and then generating a color image (color map) based on a variety of indexes including such as a tilt, a time to the peak and an area of such a graph. And when the method described in the patent Document 1, the color image is the image in which the color thereof continuously varies corresponding to e.g., the blood flow condition of the subject. A medical doctor can find the area where the blood flow condition is impaired referring to such a color image, i.e., the region where should be surgically treated.

### RELATED PRIOR ART

### Patent Document

Patent Document 1: JP Patent 5918532 B1

### ASPECTS AND SUMMARY OF THE INVENTION

### Objects to be Solved

Whereas when the method described in the patent Document 1 is applied, the time-course change of the fluorescence intensity appears as the time-course change of the pixel values in the color image and it is hard for the medical doctor to determine whether a cause for such a change is due to a change of the blood flow condition (blood circulation) or due to a halation.

The purpose of the present invention is to solve the above problem and to provide an imaging device and the method of the imaging (using the same) by which the cause can be accurately decided when the pixel values of each pixel in the predetermined region of the fluorescence image varies with time.

### Means for Solving the Problem

According to the first aspect of the present invention, an imaging device comprises: an excitation light source that irradiates an excitation light to a subject to excite a fluorochrome administered into the subject; an imaging element that acquires a fluorescence image by imaging the fluorescence emitted from the fluorochrome; an image storage element that stores the fluorescence image with time; a pixel value measurement element that measures a pixel value of each pixel of the fluorescence image; an average pixel value calculation element that calculates a first average pixel value of an entire area of the fluorescence image and a second average pixel value of a setup region of interest in the fluorescence image based on the pixel value measured with the pixel value measurement element; a first curve generation element that generates a first curve showing a time-course change of the first average pixel value of the entire area of the fluorescence image; a second curve generation element that generates a second curve showing a time-course change of the second average pixel value of the target area; and an image display unit that displays simultaneously both the first curve and the second curve.

According to the second aspect of the present invention, a method of imaging comprises: a step of irradiating an excitation light a subject to excite a fluorochrome administered to the subject; a step of acquiring a fluorescence image by imaging a fluorescence emitted from the fluorochrome; a step of storing the fluorescence image with time; a step of measuring a pixel value of respective pixels of the fluorescence with time based on a stored fluorescence image; a step of calculating a first average pixel value of an entire area of the fluorescence image based on a measured pixel value and a second average pixel value of a setup region of interest in the fluorescence image; a step of generating a time-course change of the first average pixel value of the entire fluorescence image as a first curve; a step of generating a time-course change of the second average pixel value of the entire fluorescence image as a second curve; a step of displaying the first curve and the second curve simultaneously.

### Effects of the Present Invention

According to the present invention, the time-course change of the pixel value of respective pixels of the predetermined area of the fluorescence image and the cause of the time-course change thereof can be exactly found.

The above and other aspects, features, objects, and advantages of the present invention will become apparent from the following description read in conjunction with the accompanying drawings, in which like reference numerals designate the same elements.

### BRIEF DESCRIPTION OF THE DRAWINGS

FIG. 1 is the schematic perspective view of the imaging device according to the present invention.

FIG. 2 is the schematic side view of the imaging device illustrated in FIG. 1.

FIG. 3 is the schematic plan view of the imaging device illustrated in FIG. 1.

FIG. 4 is the schematic perspective view of a lighting-imaging element of the imaging device illustrated in FIG. 1.

FIG. 5 is the schematic view of the imaging element of the lighting-imaging element illustrated in FIG. 4.

FIG. 6 is the block diagram illustrating the main control system of the imaging device illustrated in FIG. 1.

FIG. 7 is a flow diagram illustrating respective steps conducted in order using the imaging device illustrated in FIG. 1.

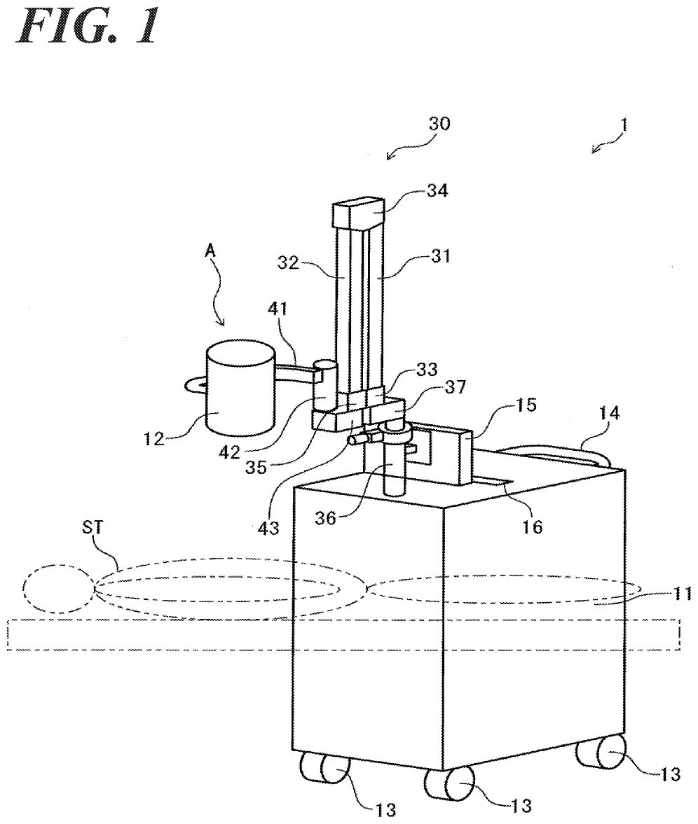

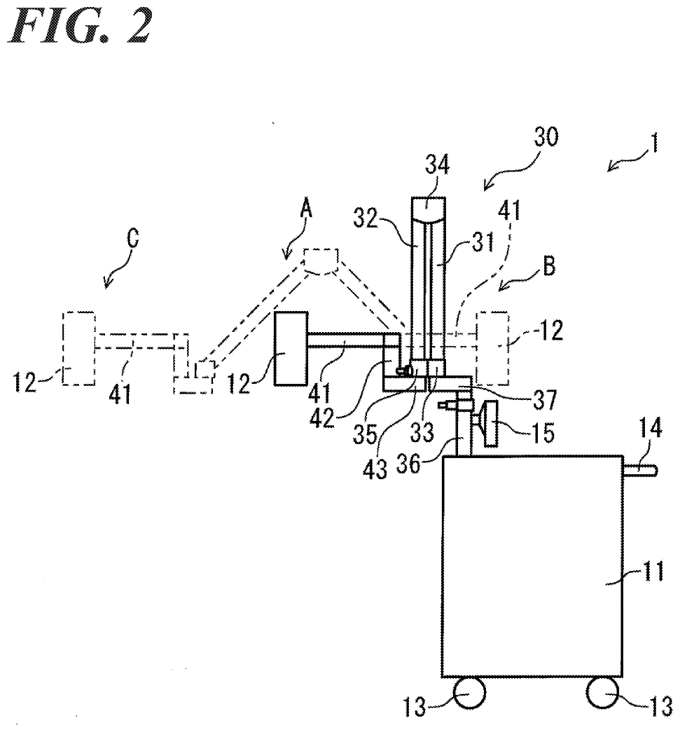

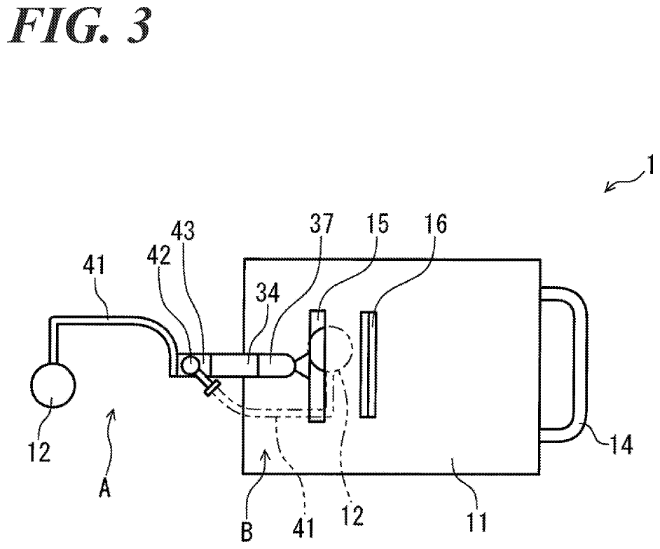

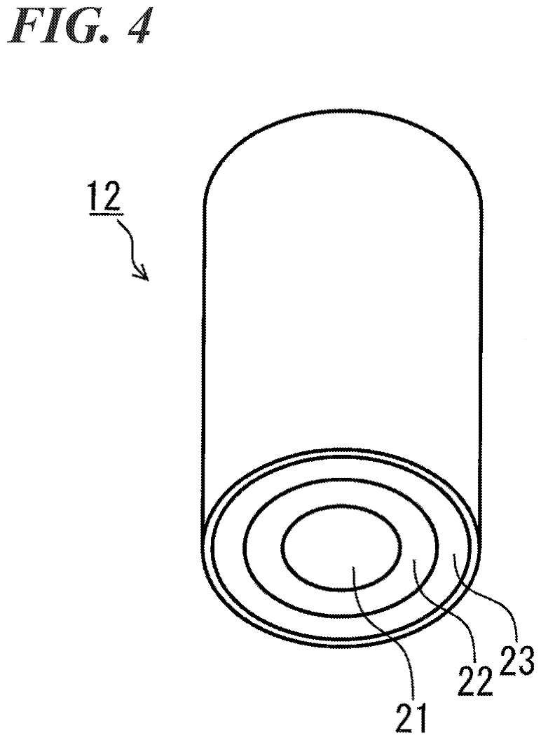

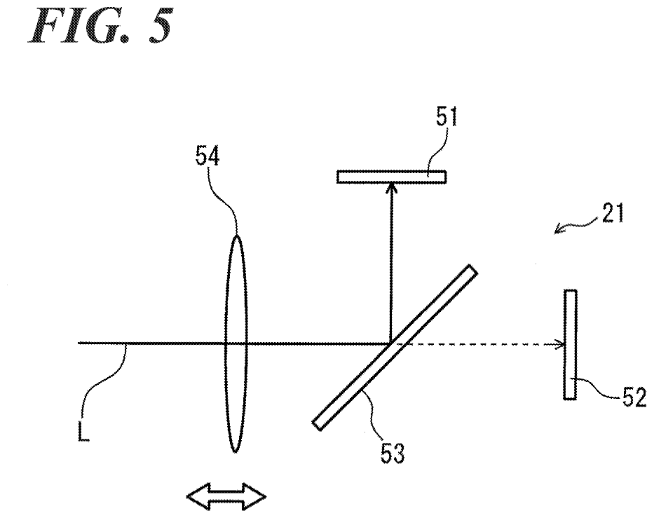

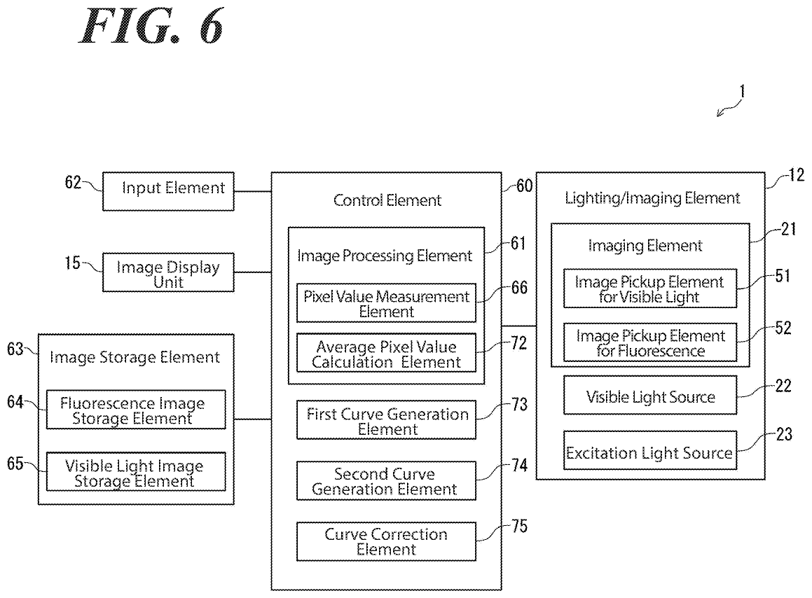

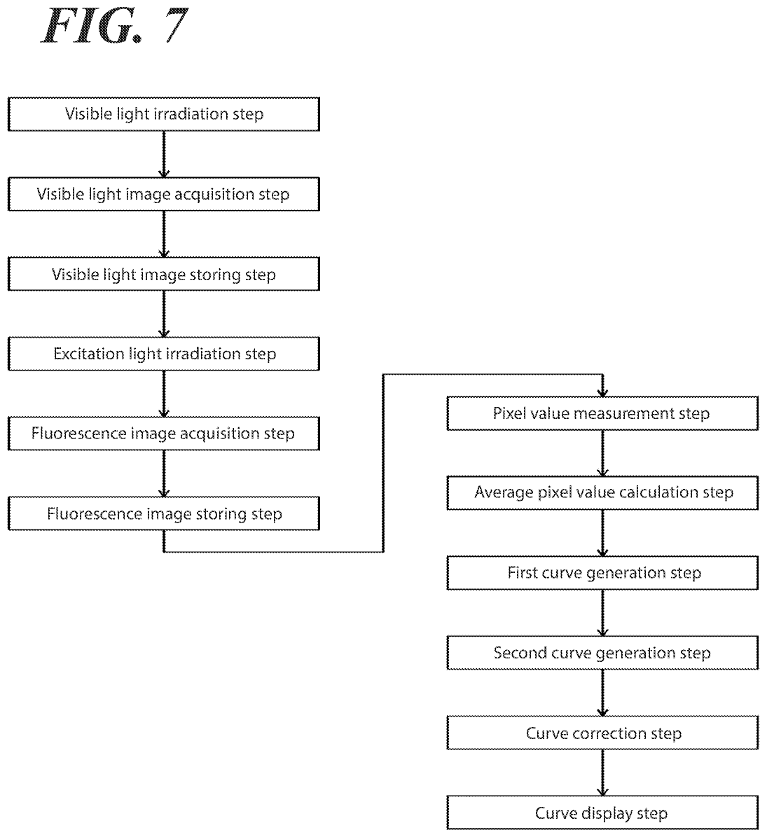

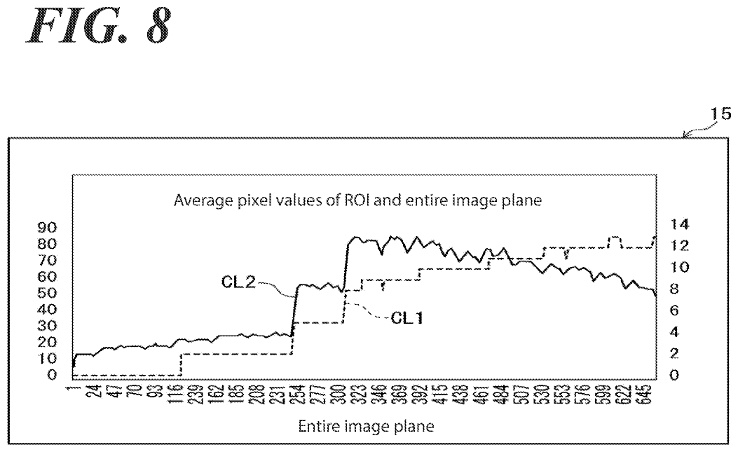

FIG. 8 is an example image obtained by conducting the steps illustrated in FIG. 7.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Hereinafter, the inventor sets forth an imaging device and a method of imaging thereof of the present invention in detail based on a preferred Embodiment based on the accompanying diagrams.

FIG. 1 is the schematic perspective view of the imaging device according to the Embodiment of the present invention. FIG. 2 is the schematic side view of the imaging device illustrated in FIG. 1. FIG. 3 is the schematic plan view of the imaging device illustrated in FIG. 1. FIG. 4 is the schematic perspective view of a lighting-imaging element of the imaging device illustrated in FIG. 1. FIG. 5 is the schematic view of the imaging element of the lighting-imaging element illustrated in FIG. 4. FIG. 6 is the block diagram illustrating the main control system of the imaging device illustrated in FIG. 1. FIG. 7 is a flow diagram illustrating respective steps conducted in order using the imaging device illustrated in FIG. 1. FIG. 8 is an example image obtained by conducting the steps illustrated in FIG. 7. In addition, hereinafter for the sake of explanation, the upper side of FIG. 1. FIG. 2 and FIG. 4 is called as top (upward) and the lower side is called as bottom (downward).

The imaging device referring to FIG. 1 irradiates an excitation light toward indocyanine green (ICG) as a fluorochrome administered into the body of the subject ST and images the fluorescence radiated from the indocyanine green. The imaging device 1 is used, so that, as set forth later, e.g., the blood flow condition of the subject ST can be exactly followed when the surgical operation is conducted on the subject ST.

The imaging device 1 comprises a wheeled platform 11 having four wheels 13, an arm mechanism 30 installed on the upper side of wheeled platform 11 and nearby the front side (left direction in FIG. 2, FIG. 3) of the wheeled platform 11 in the traveling direction thereof, a lighting-imaging element 12 installed to the arm mechanism 30 via the sub-arm 41, and a monitor 15 having the image display unit 15. A handle 14, which is used when the wheeled platform 11 is moved, is installed to the rear of the wheeled platform in the traveling direction thereof. Further, a concave portion 16 on the upper side of the wheeled platform 11 is formed to mount a remote controller to operate remotely the imaging device 1.

Further, referring to FIG. 7, the imaging device 1 can conduct the imaging using the method including a step of irradiating the visible light, a step of acquiring a visible light image, a step of storing the visible light image, a step of irradiating the excitation light, a step of acquiring the fluorescence image, a step of storing the fluorescence image, a step of measuring the pixel value, a step of calculating the average pixel value, a step of generating the first curve, a step of generating the second curve, a step of correcting the curve and a step of displaying the curve, in such an order.

The arm mechanism 30 set forth above is installed to the front-side of the wheeled platform 11 in the traveling direction thereof. Such an arm mechanism 30 comprises the first arm member 31 connected to the support element 37 installed on the support pole 36, which is installed and standing in the front-side of the wheeled platform 11 in the traveling direction thereof, with a hinge 33. The first arm member 31 is swingable relative to the wheeled platform 11 via the support pole 36 and the support element 37 due to the action of the hinge 33. In addition, the motor 15 set forth above is attached to the support pole 36.

The second arm member 32 is connected to the upper end of the first arm member 31 with a hinge 34. The second arm member 32 is swingable relative to the first arm member 31 due to the action of the hinge 34. Accordingly, referring to FIG. 2, as indicated by the virtual line (chain double-dashed line) having the reference sign C, the imaging position in which the first arm member 31 and the second arm member 32 open at the predetermined angle relative to the hinge 34, which is the connection element of the first arm member 31 and the second arm member 32, as the center, and the waiting position, indicated by the solid line in FIG. 1-FIG. 3 having the reference sign A, in which the first arm member 31 and the second arm member 32 are nearby each other, can be provided.

The support element 43 is connected to the bottom end of the second arm member 32 with a hinge 35. The support element 43 is swingable relative to the second arm member 32 due to the action of the hinge 35. The support element 43 supports the revolving axis 42. Then, the sub-arm 41 supporting the lighting-imaging element 12 revolves around the revolving axis 42 as the center, which is installed to the tip of the second arm member 32. Therefore, the lighting-imaging element 12 moves along with revolving of the sub-arm 41 between the front-side location of the wheeled platform 11 in the traveling direction thereof relative to the arm mechanism 30 to take an imaging posture or a waiting posture as indicated by the solid line with the reference sign A in FIG. 1-FIG. 3 or the virtual line (chain double-dashed line) in FIG. 2 with the reference sign C, and the rear-side location of the wheeled platform 11 in the traveling direction thereof relative to the arm mechanism 30, as indicated by the virtual line with the reference sign B in FIG. 2 and FIG. 3.

Referring to FIG. 4, the lighting-imaging element 12 further comprises an imaging element 21, which is a camera having a plurality of image pickup elements capable of imaging visible light and near-infrared light, a visible light source 22 installed in the circumference of the imaging element 21 and a light source 23 installed in the circumference of the visible light source 22.

The visible light source 22 irradiates the visible light e.g., white-light and so forth. Accordingly, a step of irradiating the visible light can be conducted, wherein the visible light is irradiated to the subject ST.

The excitation light source 23 irradiates the excitation light to excite the fluorochrome. Accordingly, a step of irradiating the excitation light can be conducted, wherein the excitation light is irradiated to the subject ST to whom the fluorochrome is administered. When the fluorochrome is an indocyanine green, it is preferable that the near-infrared light having e.g., the wavelength 810 nm is applied as the excitation light for exciting such an indocyanine green. The indocyanine green, to which the near-infrared light having the wavelength 810 nm is irradiated, emits a near-infrared light as fluorescence having a peak wavelength approximately 845 nm.

In addition, according to the present Embodiment, the lighting-imaging element 12 is applied, in which the visible light source 22, the excitation light source 23 and the imaging element 21 are unified, but the visible light source 22, the excitation light source 23 and the imaging element 21 can be independently arranged and installed.

Referring to FIG. 5, the imaging element 21 further comprises a movable lens 54 that reciprocates to focus, a wavelength selection filter 53, the imaging pickup element 51 for visible light and the imaging pickup element 52 for excitation light. the imaging pickup element 51 for visible light and the imaging pickup element 52 for excitation light are made of CMOS or CCD. Relative to the imaging system 21, the visible light and the fluorescence light are coaxially incident along the light axis thereof reach to a wavelength selection filter 53 after passing through the movable lens 54 forming the focal adjustment mechanism.

The visible light and the fluorescence pass coaxially through the wavelength selection filter 53 and are incident on the imaging pickup element 52 for fluorescence. Accordingly, a step of acquiring the fluorescence image, which acquires the fluorescence image of the subject ST, can be conducted by imaging the fluorescence emitted from the fluorochrome.

Further, the visible light of the visible light and the fluorescence incident coaxially is reflected from the wavelength selection filter 53 and is incident on the imaging pickup element 51 for visible light. Accordingly, a step of acquiring the visible light image, which acquires the visible light image of the subject ST, can be conducted by real-time imaging the visible light image in the region corresponding to the fluorescence image. And the fused image of the visible light image and the fluorescence image is displayed on the image display unit 15.

Further, with regard to the lighting-imaging element 12, the visible light is focused on the imaging pickup element 51 for visible light due to the action of the focusing mechanism including the movable lens 54, and the fluorescence is focused on the imaging pickup element 52.

The conduct order of the step of acquiring the visible light image and the step of acquiring the fluorescence image is illustrated in FIG. 7, i.e., the step of acquiring the visible light image is followed by the step of acquiring the fluorescence image, but such an order is not limited thereto and e.g., the step of acquiring the fluorescence image can be reversely followed by the step of acquiring the visible light image, or the step of acquiring the visible light image and the step of acquiring the fluorescence image can be conducted in synchronism with each other.

Referring to FIG. 6, the imaging device further comprises a CPU that executes the logic operation, a ROM that stores operation programs required to control the device, a RAM that stores temporally the data and so forth when controlling, and so forth, and further a control element 60 that controls the entire device. The control element 60 further comprises an image processing element 61 that executes various kinds of imaging processing on the visible light image and the fluorescence image. The image processing element 61 further comprises a pixel value measurement element 66 that measures respective pixel values of the predetermined area of the fluorescence image with time.

Here, “the predetermined area of the fluorescence image” is the area generally called ROI (Region of Interest). According to the imaging device 1, such as the position and size (area) of the ROI can be arbitrary set up and changed by operating the input element 62. In addition, the ROI can be the entire area of the screen of the fluorescence image or a part of the fluorescence image. Given the former, i.e., the case in which the ROI is the entire area of the screen of the fluorescence image, is selected, for example, an oversight of the blood flow condition depending on the case of the subject ST can be prevented. Given the later, i.e., the case in which the ROI is a part of the fluorescence image, is selected, for example, the image processing speed at the image processing element 61 can be increased.

The fluorochrome that is administered into the subject ST would not stay inside the ROI as-is and be flowed in some case, or in the other opposite case, almost stays in the part inside the ROI. The pixel value of respective pixels of the predetermined area of the fluorescence image, i.e., inside the ROI, is a variable value while correlated to the concentration of the fluorochrome inside the ROI. The pixel value measurement element 66 is capable of conducting the step of measuring a pixel value of each pixel of the ROI with time based on the fluorescence image stored in the image storing element 63.

Further, the imaging device 1 further comprises an image storing element 63 that stores images imaged with time using the imaging element 21. The image storage element 63 is connected to the control element 60. The image storing element 63 further comprises a fluorescence image storing element 64 that stores the fluorescence image with time and a visible light image storing element 65 that stores the visible image with time. In addition, instead of comprising the fluorescence image storing element 64 and the visible light image storing element 65, the image storing element 63 may comprise a fused image storing element that stores the fused images with time.

The step of storing the visible light image is the step of storing the visible light image with time and conducted with the visible light storing element 65.

The step of storing the fluorescence image is the step of storing the fluorescence image with time and conducted with the fluorescence image storing element 64. And the pixel value measurement element 66 is capable of measuring the pixel value of each pixel of the ROI of the fluorescence image with time based on the fluorescence image stored in the image storing element 63 as set forth above (step of measuring the pixel value).

Further, the control element 60 is connected to the input element 62 in which a variety of information is input by the operator. Further, the control element 60 is connected to the image display unit 15 set forth above. Accordingly, the image display unit 15 can display such as the fused image. In addition, the input element 62 may be installed to a remote controller that operates the imaging device 1 remotely, to the screen of the image display unit 15 when the image display unit 15 is a touch panel, or to the wheeled platform 11.

Further, the control element 60 is connected to the lighting-imaging element 12 comprising the imaging element 21, the visible light source 22 and the excitation light source 23. Accordingly, lighting-imaging element 12 can conduct the step of irradiating the visible light, the step of acquiring the visible light image, the step of irradiating the excitation light and the step of acquiring the fluorescence image.

As set forth before, the pixel value of each pixel of the ROI varies with time along with the change of the blood flow condition. However, the time-course change of such a pixel may be caused by a halation besides the change of the blood flow condition. It is hard for the medical doctor to determine whether a cause for such a time-course change is due to a change of the blood flow condition or due to the halation.

Therefore, the imaging device 1 is configured to be capable of finding accurately the cause thereof when the pixel value of each pixel of the ROI changes with time. Hereinafter. the inventor sets forth such a structure and action. Hereinafter. the inventor sets forth such a structure and action.

Referring to FIG. 6, the control element 60 comprises an average pixel value calculation element 72, a first curve generation element 73, a second curve generation element 74 and a curve correction element 75.

The average pixel value calculation element 72 is built into the image processing element 61. The average pixel value calculation element 72 calculates an average pixel value of the entire area of the fluorescence image and an average pixel value of ROI that is set up in the fluorescence image based on the pixel value measured with the pixel value measurement element 66.

Here, the method of calculating the average pixel value of the entire area of the fluorescence image is not particularly limited, and for example, the method of dividing the total of the pixel value of the pixel in the entire area of the fluorescence image by the number of pixels thereof every predetermined time (every 24 seconds when obtaining the graph in FIG. 8), i.e., isochronous duration, can be adopted.

Also, the method of calculating the average pixel value of the ROI is not particularly limited, and for example, the method of dividing the total of the pixel value of the ROI by the number of pixels thereof every predetermined time, which is the same as when calculating the average pixel value of the entire area of the fluorescence image, can be applied.

In such way, the imaging device 1 conducts the step of calculating the average pixel value of an entire area of the fluorescence image and the average pixel value of the setup ROI in the fluorescence image based on the pixel value measured with the pixel value measurement element 66. Accordingly, the time-course change of the average pixel value in the entire area of the fluorescence image and the time-course change of the average value of the ROI can be obtained.

And the first curve generation element 73 generates the time-course change of the average pixel value of the entire area of the fluorescence image as a first curve CL1. Further, the second curve generation element 74 generates the time-course change of the average pixel value of the ROI as a second curve CL2. The method of generating the respective curves is not limited, e.g., a TLC (Time Intensity Curve) analysis can be applied.

According to the graph referring to FIG. 8, the horizontal axis denotes the time, the left vertical axis denotes an average pixel value of the ROI and the right vertical axis denotes average pixel value of the entire area of the fluorescence image.

In such way, the imaging device 1 conducts the step of generating the first curve to make the first curve CL1 representing the time-course change of the average pixel value of the entire area of the fluorescence image following the step of calculating the average pixel value and the step of generating the second curve to make the second curve CL2 representing the time-course change of the average pixel value of the ROI.

A curve correction element 75 corrects the second curve CL2 based on the time differential value of the first curve CL1. Here, “correction” means that when the time differential value is higher than the predetermined value, the average pixel value of the ROI at the time corresponding to the time differential value is multiplied by the predetermined value based on the time differential value to correct the second curve CL2.

For example, referring to FIG. 8, it can be understood that not only the second curve CL2 (i.e., the average pixel value of the ROI) largely varies the time (elapsed time) of 254 seconds and 300 seconds, but also at the same time, the first curve CL1 (i.e., the average pixel value of the entire area of the fluorescence image) varies largely (i.e., the time differential value is large). In such way, the fact, in which not only the second curve CL2, but also the first curve CL1 changes largely, implies that the halation takes place on the entire fluorescence image and the effect thereof is dominant. Then, referring to FIG. 8, the time (elapsed time) at 254 seconds and 300 seconds, the time differential value (i.e., variation) of the first curve CL1 is obtained and the value of the second curve CL2 at the same time is multiplied by such a number and the predetermined coefficient, In such way, once the value of the second curve CL2 is corrected, the effect of the halation on the second curve CL2 is erased or reduced, so that the corrected second curve CL2 represents almost the change of the condition of the blood flow.

In such way, the curve correction element 75 conducts the step of correcting the curve, in which the second curve CL2 is corrected based on the time differential value of the first curve CL1. Accordingly, the corrected second curve CL2 showing almost the change of the condition of the blood flow can be obtained. In addition, the curve correction step (curve correction element 75) is not the mandatory step and can be skipped.

Referring to FIG. 8, the image display unit 15 displays simultaneously the first curve CL1 and the second curve CL2. Accordingly, the step of displaying simultaneously the first curve CL1 and the second curve CL2 is conducted. In addition, referring to FIG. 8, the second curve CL2 generated at the step of generating the second curve is shown, but the second curve CL2 after correction, which is corrected at the step of correcting the curve (curve correction element 75), can be displayed as well.

As set forth above, the change of the second curve CL2 shows essentially the change of the blood flow, but once the halation takes place, the effect thereof affects the second curve CL2, so that the image display unit 15 displays simultaneously the first curve CL1 and the second curve CL2 in the present Embodiment. Specifically, referring to FIG. 8, when not only the second curve CL2 but also the first curve CL1 change largely at 254 seconds and 300 seconds, the cause for the change of the second curve CL2 can be found due to the halation. And in such way, when the effect due to the halation is superimposed to the second curve CL2, the medical doctor discretionary measures the ROI again.

As set forth above, according to the present Embodiment, it can be determined easily and accurately that the change of the second curve CL2 (i.e., the average pixel value of the ROI) is due to the change of the blood flow or the halation. Therefore, the medical doctor can perform such as the safe, accurate and adequate surgery on the subject ST.

As set forth above, the inventor illustrates the imaging device and the method of imaging according to Embodiment referring to FIGs, but the present invention is not limited thereto. Further, respective elements of the imaging device can be replaced with any arbitrary element that can provide the same function. Further, an arbitrary component may be added.

Further, according to the present Embodiments set forth above, the inventors set forth the case in which indocyanine green is applied as a material containing a fluorochrome and the near-infrared light having the wavelength 600 nm to 850 nm is irradiated toward the indocyanine green as an excitation light and then the fluorescence in the near-infrared region, having the peak wavelength approximately 810 nm, from indocyanine green is emitted, but a light other than near-infrared light can be applied.

Further, e.g., 5-ALA/5-aminolevulinic acid may be applied instead of indocyanine green as the fluorochrome depending on the case of the subject.

(Aspect)

A plurality of Embodiments set forth above is understood as the specific Embodiments by a person skilled in the art.

(Term 1) An imaging device 1 according to one aspect of the present invention comprises: an excitation light source 23 that irradiates a subject with an excitation light that excites a fluorochrome administered into the subject; an imaging element 21 that acquires a fluorescence image by imaging the fluorescence emitted from the fluorochrome; an image storing element 63 that stores the fluorescence image with time; a pixel value measurement element 66 that measures a pixel value of each pixel of the fluorescence image with time based on the fluorescence image stored in the image storing element; an average pixel value calculation element 72 that calculates an average pixel value of an entire area of the fluorescence image and an average pixel value of a region of interest setup in the fluorescence image based on the pixel value measured with the pixel value measurement element; a first curve generation element 73 that generates a first curve showing a time-course change of the average pixel value of the entire area of the fluorescence image; a second curve generation element 74 that generates a second curve showing a time-course change of the average pixel value of the region of interest; and an image display unit 15 that displays simultaneously the first curve and the second curve.

According to the imaging device in Term 1, it can be determined easily and accurately that the change of the second curve is whether due to a change of a blood flow or a halation.

(Term 2) The imaging device according to Term 1 further comprises a curve correction element 75 that corrects the second curve based on the time differential value of the first curve.

The imaging device according to Term 2 can erase or reduce an effect of the halation relative to the second curve.

(Term 3) The imaging device according to Term 2, wherein the curve correction element 75 can correct the second curve with multiplying the average pixel value of the region of interest by the predetermined value based on the time differential value when the time differential value is higher than a predetermined value.

The imaging device according to Term 3, only the effect of the halation relative to the second curve can be accurately erased or reduced.

(Term 4) A method of imaging according to one aspect of the present Embodiment comprises: a step of irradiating a subject with an excitation light that excites a fluorochrome administered into the subject; a step of acquiring a fluorescence image by imaging the fluorescence emitted from the fluorochrome; a step of storing the fluorescence image with time; a step of measuring a pixel value of each pixel of the fluorescence image with time based on the fluorescence image that is stored therein; a step of calculating an average pixel value of an entire area of the fluorescence image and an average pixel value of a region of interest setup in the fluorescence image based on the pixel value that is measured therewith; a step of generating a first curve showing a time-course change of the average pixel value of the entire area of the fluorescence image; a step of generating a second curve showing a time-course change of the average pixel value of the region of interest; and a step of displaying simultaneously the first curve and the second curve.

According to the imaging device in Term 4, it can be determined easily and accurately that the change of the second curve is whether due to a change of a blood flow or a halation.

(Term 5) With regards to the method according to Term 4 further comprises a step of correcting the second curve based on the time differential value of the first curve.

The imaging device according to Term 5 can erase or reduce the effect of the halation relative to the second curve.

(Term 6) According to the method according to Term 5, further comprises the step of correcting the second curve can correct the second curve by multiplying the average pixel value of the region of interest by the predetermined value based on the time differential value at the time corresponding to the time differential value when the time differential value is larger than the predetermined value.

The imaging method according to Term 6 can accurately erase or reduce only the effect of the halation relative to the second curve.

### REFERENCE OF SIGNS

1 Imaging Device
11 Wheeled platform
12 Lighting-imaging element
13 Wheel
14 Handle
15 Image display unit
16 Concave element
21 Imaging element
22 Visible light source
23 Excitation light source
30 Arm mechanism
31 First arm member
32 Second arm member
33 Hinge element
34 Hinge element
35 Hinge element
36 Supporting post
37 Support unit
41 Sub-arm
42 Revolving axis
43 Support unit
51 Visible light pickup element
52 Fluorescence light pickup element
53 Wavelength selection filter
54 Movable lens
60 Control element
61 Image processing element
62 Input element
63 Image storing element
64 Fluorescence image storing element
65 Visible light image storing element
66 Pixel value measurement element
72 Average pixel value calculation element
73 First curve generation element
74 Second curve generation element
75 Curve correction element
CL1 First curve
CL2 Second curve
ST Subject

Also, the inventors intend that only those claims which use the specific and exact phrase “means for” are intended to be interpreted under 35 USC 112, sixth paragraph. The structure herein is noted and well supported in the entire disclosure. Moreover, no limitations from the specification are intended to be read into any claims, unless those limitations are expressly included in the claims.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. An imaging device, comprising:
an excitation light source that irradiates a subject with an excitation light that excites a fluorochrome administered into said subject;
an imaging element that acquires a fluorescence image by imaging a fluorescence emitted from said fluorochrome;
an image storing element that stores said fluorescence image with time;
a pixel value measurement element that measures a pixel value of each pixel of said fluorescence image with time based on said fluorescence image stored in said image storing element;
an average pixel value calculation element that calculates a first average pixel value of an entire area of said fluorescence image and a second average pixel value of a region of interest setup in said fluorescence image based on said pixel value measured with said pixel value measurement element;
a first curve generation element that generates a first curve showing a time-course change of said first average pixel value of said entire area of said fluorescence image;
a second curve generation element that generates a second curve showing a time-course change of said second average pixel value of said region of interest; and
an image display unit that displays simultaneously said first curve and second curve.

2. The imaging device, according to claim 1, further comprising:
a curve correction element that corrects said second curve based on a time differential value of said first curve.

3. The imaging device, according to claim 2, wherein:
said curve correction element corrects said second curve by multiplying said second average pixel value of said region of interest by a first predetermined value based on said time differential value when said time differential value is larger than a second predetermined value.

4. An imaging method, comprising:
a step of irradiating a subject with an excitation light that excites a fluorochrome administered into the subject;
a step of acquiring a fluorescence image by imaging a fluorescence emitted from said fluorochrome;
a step of storing said fluorescence image with time;
a step of measuring a pixel value of each pixel of said fluorescence image with time based on said fluorescence image that is stored therein;
a step of calculating a first average pixel value of an entire area of said fluorescence image and a second average pixel value of a region of interest setup in said fluorescence image based on said pixel value that is measured therewith;
a step of generating a first curve showing a time-course change of said first average pixel value of the entire area of said fluorescence image;
a step of generating a second curve showing a time-course change of said second average pixel value of said region of interest; and
a step of displaying simultaneously said first curve and said second curve.

5. The imaging method, according to claim 4, further comprising:
a step of correcting said second curve based on a time differential value of said first curve.

6. The imaging method, according to claim 1, wherein:
said step of correcting said second curve with multiplying said second average pixel value of said region of interest by said first predetermined value based on said time differential value when said time differential value is larger than said second predetermined value.
